(19) United States
(12) Patent Application Publication
(10) Pub. No.: US 2011/0171698 A1
(43) Pub. Date: Jul. 14, 2011

(54) C-22 HYDROXYLASE

(75) Inventors: Yutaka Ebizuka, Yokohama-shi (JP); Masaaki Shibuya, Bunkyo-ku (JP); Eriko Wakita, Tatebayashi-shi (JP)

(73) Assignee: MEIJI SEIKA KAISHA, LTD., Chuo-ku (JP)

(21) Appl. No.: 12/690,732

(22) Filed: Jan. 20, 2010

(30) Foreign Application Priority Data
Jul. 28, 2009 (JP) 2009-175880

(51) Int. Cl. C12P 15/00; C07H 21/00; C07H 21/04; C12N 15/63; C12N 5/10; C12N 1/00; C12N 1/19; C12N 9/02

(52) U.S. Cl. 435/127; 536/23.2; 435/320.1; 435/419; 435/325; 435/243; 435/254.2; 435/189

(57) ABSTRACT

β-Amyrin, a precursor in biosynthesis of soyasapogenol B, is biosynthesized by cyclization of 2,3-oxidosqualene which is generated by the mevalonate pathway, and soyasapogenol B is biosynthesized by two hydroxylations of β-amyrin. However, a gene of 22-hydroxylase involved in the sequence of reactions has not been identified. The present inventors identified a gene encoding the hydroxylase for oleanene triterpenes at C-22, and found that oleanene triterpenes could be hydroxylated at C-22 by co-expressing this gene together with one or more specific genes. Further, the present inventors found that soyasapogenol B could be efficiently produced in large quantities by co-expressing this gene for 22-hydroxylase with a gene for 24-hydroxylase.

### TECHNICAL FIELD

The present invention relates to an enzyme which is derived from a plant and is involved in soyasapogenol B biosynthesis, its gene, and a use thereof.

### BACKGROUND ART

Triterpene saponins are a group of compounds contained in various medicinal and edible plants, and are typical secondary metabolites with steroid or triterpenoid skeletons to which sugar molecules are added by ether or ester bonds. The biosynthesis of triterpene saponins involves cyclization of 2,3-oxidosqualene, which is a C30 isoprenoid derived from mevalonate and a common precursor of all the cyclizations, followed by oxidative modification of the skeletons and transfer of the sugar moiety. It is considered that a wide range of structural diversity is produced by this sequence of reactions.

Many enzymes which catalyze the cyclization, the initial step of biosynthesis of triterpene saponins, have been cloned (non-patent literatures 2 and 3) on the basis of homologies to 2,3-oxidosqualene-lanosterol cyclases (non-patent literature 1), which have been intensively studied in steroid metabolic pathways, and recent developments in molecular biology.

The subsequent oxdative modification produces further structural diversity of triterpenes. However, because of difficulties in detecting oxidase activities, almost all of the detailed mechanisms have not been revealed. There are only a few reports that oxidase activities for a triterpene or its analogue, a secondary metabolite-type steroid, were detected in an in vitro enzyme reaction experiment or an in vitro administration experiment using a plant body or cultured cells (non-patent literatures 4 and 5).

Because many oxidases such as P450 enzymes are membrane-bound enzymes which require other factors such as NADPH and FMN, it is difficult to purify these enzyme proteins due to their low stability. Therefore, it is very difficult to clone triterpene oxidases by conventional methods, i.e., detecting an enzyme activity at the level of a plant body or cultured cells, purifying the enzyme, and identifying a gene for biosynthesis on the basis of the information.

Glycine max belonging to Leguminosae produces many types of triterpene saponins known as soyasaponins (non-patent literatures 6 and 7). These soyasaponins are mainly classified into two groups A and B according to their aglycon structures. The aglycon moiety of group B saponins is soyasapogenol B (olean-12-ene-3β,22β,24-triol), a hydroxylated product of β-amyrin (olean-12-ene-3β-ol) at C-22 and C-24. This indicates the presence of an enzyme which hydroxylates β-amyrin in Glycine max. It is considered that soyasapogenol B is biosynthesized via two hydroxylations of β-amyrin. Soyasapogenol B is finally converted into soyasaponins such as soyasaponin I by a sugar transfer reaction.

With respect to the hydroxylation of β-amyrin at the C-24 position, the present inventors identified an enzyme (CYP93E1) which hydroxylates β-amyrin and sophoradiol (olean-12-ene-3β,22β-diol) at C-24 from Glycine max using a functional genomic approach (non-patent literature 8 and patent literature 1).

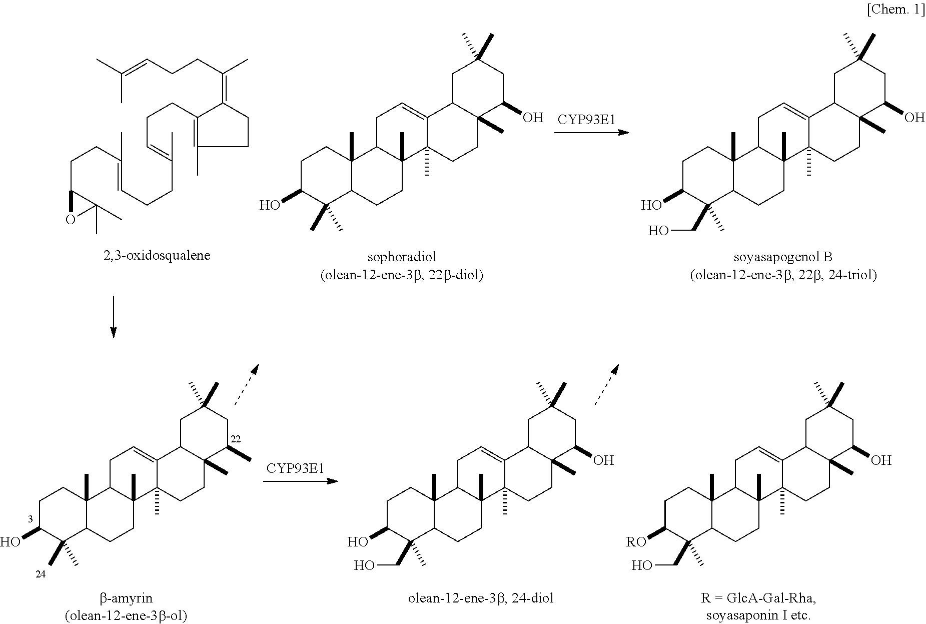

### CITATION LIST

### Patent Literature

[patent literature 1] WO 2005/080572

### Non-Patent Literature

[non-patent literature 1] E. J. Corey, S. P. T. Matsuda, B. Bartel, Proc. Natl. Acad. Sci. USA (1994) 91, 2211-2215
[non-patent literature 2] E. J. Corey, S. P. T. Matsuda, B. Bartel, Proc. Natl. Acad. Sci. USA (1993) 90, 11628-11632
[non-patent literature 3] J. B. R. Herrera et al., Phytochem. (1998) 49, 1905-1911
[non-patent literature 4] H. Hayashi et al., Phytochem. (1993) 34, 1303-1307
[non-patent literature 5] M. Peterson, H. U. Seitz, FEBS Lett. (2006) 1985, 188, 11-14
[non-patent literature 6] S. Kudou et al., Biosci. Biotech. Biochem. (1993) 57, 546-550
[non-patent literature 7] M. Shiraiwa et al., Agric. Biol. Chem. (1991) 55, 315-322
[non-patent literature 8] M. Shibuya, M. Hoshino, Y. Katsube, H. Hayashi, T. Kushiro, Y. Ebizuka, FEBS J. (2006) 273, 948-959

### SUMMARY OF INVENTION

### Technical Problem

β-Amyrin, a precursor in biosynthesis of soyasapogenol

B, is biosynthesized by cyclization of 2,3-oxidosqualene which is generated by the mevalonate pathway, and soyasapogenol B is biosynthesized by two hydroxylations of β-amyrin. However, the gene of a hydroxylase for oleanene triterpenes at C-22 involved in the sequence of reactions has not been identified. Therefore, the hydroxylase for oleanene triterpenes at C-22 cannot be used by means of gene engineering.

### Solution to Problem

The present inventors conducted intensive studies to solve the problem and, as a result, identified a gene encoding the hydroxylase for oleanene triterpenes at C-22, and found that this gene hydroxylated oleanene triterpenes at C-22. Further, the present inventors found that soyasapogenol B could be efficiently produced in large quantities by co-expressing the gene for 22-hydroxylase with a gene for 24-hydroxylase.

The present invention provides as follows:

1. A polynucleotide encoding a polypeptide having 22-hydroxylase activity for an oleanene triterpene.
2. The polynucleotide of 1, which is a DNA selected from the group consisting of:
(a) a DNA encoding a polypeptide comprising (preferably, consisting of) the amino acid sequence of SEQ ID NO: 2;
(b) a DNA comprising the coding region in the nucleotide sequence of SEQ ID NO: 1;
(c) a DNA encoding a polypeptide comprising (preferably, consisting of) an amino acid sequence in which one or plural amino acids are deleted, substituted, inserted, and/or added in the amino acid sequence of SEQ ID NO: 2, wherein the polypeptide has 22-hydroxylase activity for an oleanene triterpene;
(d) a DNA encoding a polypeptide comprising (preferably, consisting of) an amino acid sequence with at least 80% identity to SEQ ID NO: 2, wherein the polypeptide has 22-hydroxylase activity for an oleanene triterpene;
(e) a DNA comprising (preferably, consisting of) a nucleotide sequence with at least 80% identity to SEQ ID NO: 1, wherein a polypeptide encoded by the nucleotide sequence has 22-hydroxylase activity for an oleanene triterpene; and
(f) a DNA which hybridizes under stringent conditions to a DNA consisting of the nucleotide sequence of SEQ ID NO: 1, and encodes a polypeptide having 22-hydroxylase activity for an oleanene triterpene.
3. A recombinant vector comprising the polynucleotide of 1 or 2.
4. A transformant transformed with the recombinant vector of 3.
5. The transformant of 4, which is a yeast.
6. A polypeptide selected from the group consisting of:
(a) a polypeptide comprising (preferably, consisting of) the amino acid sequence of SEQ ID NO: 2;
(b) a polypeptide comprising (preferably, consisting of) the amino acid sequence encoded by the nucleotide sequence of SEQ ID NO: 1;
(c) a polypeptide comprising (preferably, consisting of) an amino acid sequence in which one or plural amino acids are deleted, substituted, inserted, and/or added in the amino acid sequence of SEQ ID NO: 2, wherein the polypeptide has 22-hydroxylase activity for an oleanene triterpene;
(d) a polypeptide comprising (preferably, consisting of) an amino acid sequence with at least 80% identity to SEQ ID NO: 2, wherein the polypeptide has 22-hydroxylase activity for an oleanene triterpene;
(e) a polypeptide comprising (preferably, consisting of) an amino acid sequence encoded by a nucleotide sequence with at least 80% identity to SEQ ID NO: 1, wherein the polypeptide has 22-hydroxylase activity for an oleanene triterpene; and
(f) a polypeptide encoded by a DNA which hybridizes under stringent conditions to a DNA consisting of the nucleotide sequence of SEQ ID NO: 1, wherein the polypeptide has 22-hydroxylase activity for an oleanene triterpene.
7. A process of manufacturing sophoradiol, comprising the steps of:
preparing a first recombinant vector comprising the polynucleotide of 1, and a second recombinant vector comprising a DNA encoding a polypeptide having β-amyrin synthase activity,
co-expressing the two recombinant vectors in a same host cell,
cultivating the expressed host cell, and
purifying sophoradiol from the host cell or a culture supernatant.
8. The method of 7, wherein a third recombinant vector comprising a DNA encoding a polypeptide having cytochrome P450 reductase activity is further expressed in the same host cell.
9. The method of 7 or 8, wherein the host cell is a yeast.
10. A process of manufacturing soyasapogenol B, comprising the steps of:
preparing a first recombinant vector comprising the polynucleotide of 1, a second recombinant vector comprising a DNA encoding a polypeptide having β-amyrin synthase activity, and a third recombinant vector comprising a DNA encoding a polypeptide having 24-hydroxylase activity for an oleanene triterpene,
co-expressing the three recombinant vectors in a same host cell,
cultivating the expressed host cell, and
purifying soyasapogenol B from the host cell or a culture supernatant.
11. The method of 10, wherein a fourth recombinant vector comprising a DNA encoding a polypeptide having cytochrome P450 reductase activity is further expressed in the same host cell.
12. The method of 10 or 11, wherein the host cell is a yeast.

### Effects of Invention

The present invention has revealed the gene of 22-hydroxylase for oleanene triterpenes. According to the present invention, 22-hydroxylated oleanene triterpenes can be produced in large quantities by means of gene engineering, more particularly, by direct cultivation of host cells co-expressing the specific 22-hydroxylase gene and the specific cytochrome P450 gene.

### BRIEF DESCRIPTION OF DRAWINGS

FIG. 1 illustrates the outline of the determination of the full-length sequence of soybean CYP72A61 in Example 2.

FIG. 2 schematically illustrates the structures of plasmids used to construct transformant GIL747/pESC-URA-PSY/pESC-LEU-GmCPR/pESC-HIS-CYP72A61 prepared in Example 9.

FIG. 3 shows total ion chromatograms of extracts from transformed yeast strain INVSc1 co-expressing PSY, CYP93E1, and GmCPR, measured in Example 7.

A: Acetylation product of authentic olean-12-ene-3β,24-diol

B: Extract from INVSc1/pESC-URA-PSY-CYP93E1/pESC-HIS-GmCPR cells

C: Extract from INVSc1/pESC-URA-PSY-CYP93E1/pESC-HIS cells

FIG. 4 shows MS cleavage patterns of the peak at a retention time of 9.05 minutes shown in FIG. 5.

A: Acetylation product of authentic olean-12-ene-3β,24-diol

B: Extract from INVSc1/pESC-URA-PSY-CYP93E1/pESC-HIS-GmCPR cells

C: Extract from INVSc1/pESC-URA-PSY-CYP93E1/pESC-HIS cells

FIG. 5 shows total ion chromatograms of extracts from transformed yeast strain GIL747 co-expressing PSY, GmCPR, and CYP72A61, measured in Example 9.

A: Trimethylsilylation product of authentic sophoradiol

B: Extract from GIL747/pESC-URA-PSY/pESC-LEU-GmCPR/pESC-HIS-CYP72A61 cells

C: Extract from GIL747/pESC-URA-PSY/pESC-LEU-GmCPR/pESC-HIS cells

FIG. 6 shows MS cleavage patterns of the peak at a retention time of 9.05 minutes shown in FIG. 5.

A: Trimethylsilylation product of authentic sophoradiol

B: Extract from GIL747/pESC-URA-PSY/pESC-LEU-GmCPR/pESC-HIS-CYP72A61

FIG. 7 shows MS chromatograms at m/z 306 of extracts from transformed yeast strain INVSc1 co-expressing GmCPR and CYP72A61, measured in Example 11.

A: Trimethylsilylation product of authentic soyasapogenol B

B: Extract from INVSc1/pESC-LEU-GmCPR/pESC-HIS-CYP72A61 cells

C: Extract from INVSc1/pESC-LEU-GmCPR/pESC-HIS cells

FIG. 8 shows MS cleavage patterns of the peak at a retention time of 9.85 minutes shown in FIG. 7.

A: Trimethylsilylation product of authentic soyasapogenol B

B: Extract from INVSc1/pESC-LEU-GmCPR/pESC-HIS-CYP72A61 cells

C: Extract from INVSc1/pESC-LEU-GmCPR/pESC-HIS cells

FIG. 9 schematically illustrates the structures of plasmids used to construct transformant GIL747/pESC-URA-PSY-CYP93E1/pESC-LEU-GmCPR/pESC-HIS-CYP72A61 prepared in Example 12.

FIG. 10 shows total ion chromatograms of extracts from transformed yeast strain GIL747 co-expressing PSY, CYP93E1, GmCPR, and CYP72A61, measured in Example 12.

A: Trimethylsilylation product of authentic sophoradiol

B: Trimethylsilylation product of authentic soyasapogenol B

C: Extract from GIL747/pESC-URA-PSY-CYP93E1/pESC-LEU-GmCPR/pESC-HIS-CYP72A61 cells

D: Extract from GIL747/pESC-URA-PSY-CYP93E1/pESC-LEU-GmCPR/pESC-HIS cells

FIG. 11 shows MS cleavage patterns of the peak at a retention time of 9.85 minutes shown in FIG. 10.

B: Trimethylsilylation product of authentic soyasapogenol B

C: Extract from GIL747/pESC-URA-PSY-CYP93E1/pESC-LEU-GmCPR/pESC-HIS-CYP72A61 cells

### DESCRIPTION OF EMBODIMENTS

Examples of known oleanene triterpenes include β-amyrin, sophoradiol, olean-12-ene-3β,24-diol, soyasapogenol A, and soyasapogenol B, but the term “oleanene triterpenes” as used herein is not limited to these known compounds.

Examples of oleanene triterpenes in which the C-22 position is to be hydroxylated include β-amyrin and olean-12-ene-3β,24-diol, but are not limited to these compounds, so long as the C-22 position is hydroxylated by the method of the present invention.

The present inventors selected five clones for soybean cytochrome P450 for which ESTs and functionally undefined nucleotide sequences had been reported [TIGR (The Institute for Genomic Research) Accession Number: TC204312, TC225935, TC218097, TC224852, and TC209136]. Among the clones, the polynucleotide of SEQ ID NO: 1 with high homology to CYP72A61 (GenBank Accession Number: DQ335793, TIGR Accession Number: TC204312) exhibited 22-hydroxylase activity, but the other four clones did not exhibit 22-hydroxylase activity.

In the present invention, 22-hydroxylated oleanene triterpenes can be produced by utilizing a transcription or translation product of the polynucleotide of SEQ ID NO: 1 or an equivalent thereof. The term “equivalent” as used herein means a sequence which encodes a polypeptide having the same functions (in particular, 22-hydroxylase activity for oleanene triterpenes) as those of a polypeptide consisting of the amino acid sequence encoded by the nucleotide sequence of SEQ ID NO: 1 (i.e., the amino acid sequence of SEQ ID NO: 2) and hybridizes under stringent conditions to the polynucleotide sequence complementary to SEQ ID NO: 1.

The feature “to hybridize under stringent conditions” can be confirmed by hybridizing a polynucleotide of interest to the whole or part of DNA of SEQ ID NO: 1 (or its complementary chain) as a probe. The hybridization may be carried out by, for example, colony hybridization, plaque hybridization, or Southern blot hybridization. More particularly, hybridization may, for example, be carried out at 55° C. in the presence of 0.5 mol/L sodium chloride, and then, 2×SSC solution (1×SSC solution: 150 mmol/L NaCl, 15 mmol/L sodium citrate, pH7.0) may be used.

Hybridization can be carried out in accordance with, for example, the method described in Molecular cloning: A Laboratory Manual, 2^{nd} Ed., T. Maniatis et al., Cold Spring Harbor laboratory, 1989.

The polynucleotide (in particular, DNA) which hybridizes under stringent conditions to a DNA probe may be a polynucleotide whose identity to the nucleotide sequence of the DNA probe exceeds a certain value. The term “identity” as used herein means a percentage of identical nucleotides contained in each sequence that are compared. More particularly, the polynucleotide may be a polynucleotide with at least 80% identity (preferably 90% or more, more preferably 93% or more, still more preferably 95% or more, and most preferably 98% or more) to SEQ ID NO: 1, according to the calculation using BLAST (National Center for Biotechnology Information).

Examples of the polynucleotide which hybridizes under stringent conditions to the complementary chain of the polynucleotide of SEQ ID NO: 1, and encodes a polypeptide having 22-hydroxylase activity for oleanene triterpenes include a polynucleotide which has a nucleotide sequence in which one or plural nucleotides are deleted, substituted, inserted, and/or added in the nucleotide sequence of SEQ ID NO: 1, and encodes a polypeptide having 22-hydroxylase activity for oleanene triterpenes. The number of nucleotides deleted, substituted, inserted, and/or added is not limited, so long as the polynucleotide sequence can encode a polypeptide having the desired activity.

Mutations on the polynucleotide of cytochrome P450 gene CYP72A61 include a naturally-occurring mutation and an artificial mutation. Examples of the artificial mutation include a method in which a random mutation or a site-directed mutation is introduced into the polynucleotide of cytochrome P450 gene CYP72A61, by means of gene engineering, to obtain a polynucleotide in which at least one modification selected from deletion, substitution, insertion, and addition of one or plural nucleotide residues is carried out. Such a mutated polynucleotide may be used to obtain a polypeptide having properties different from those of the original enzyme in, for example, optimum temperature, heat stability, optimum pH, pH stability, and substrate specificity.

The term “amino acid sequence in which one or plural amino acids are deleted, substituted, inserted, and/or added in a certain amino acid sequence” as used herein means that the original amino acid sequence is modified, for example, by a well-known method such as site-directed mutagenesis, or by naturally occurring substitution of several numbers of amino acids. The number of amino acids modified is preferably 1 to 50 amino acids, more preferably 1 to 30 amino acids, still more preferably 1 to 10 amino acids, still more preferably 1 to 5 amino acids, and most preferably 1 to 2 amino acids.

The term “amino acid sequence with at least 80% identity to SEQ ID NO: 2” as used herein means an amino acid sequence with at least 80% identity (preferably 85% or more, more preferably 90% or more, still more preferably 95% or more, and most preferably 98% or more) to SEQ ID NO: 2, according to the calculation using BLAST (National Center for Biotechnology Information).

Examples of modified amino acid sequences in the polypeptide of the present invention include, preferably amino acid sequences including one or several (preferably 1, 2, 3, or 4) conservative substitutions.

The term “conservative substitution” as used herein means that one or several amino acid residues are replaced with different amino acids having similar chemical properties. Examples of the conservative substitution include substitution of a hydrophobic residue for another hydrophobic residue, and substitution of a polar residue for another polar residue having the same charge. Amino acids which have similar chemical properties and can be conservatively substituted with each other are known to those skilled in the art. More particularly, examples of nonpolar (hydrophobic) amino acids include alanine, valine, isoleucine, leucine, proline, tryptophan, phenylalanine, and methionine. Examples of polar (neutral) amino acids include glycine, serine, threonine, tyrosine, glutamine, asparagine, and cysteine. Examples of basic amino acids having a positive charge include arginine, histidine, and lysine. Examples of acidic amino acids having a negative charge include aspartic acid and glutamic acid.

The polynucleotide which hybridizes under stringent conditions to the complementary chain of the polynucleotide of cytochrome P450 gene CYP72A61, and encodes a polypeptide having 22-hydroxylase activity for oleanene triterpenes may be obtained, by hybridization (for example, colony hybridization, plaque hybridization, or Southern blot hybridization) using the whole or part of a polynucleotide having the nucleotide sequence of cytochrome P450 gene CYP72A61 (or its complementary chain) as a probe, or by PCR using the whole or part of a polynucleotide having the nucleotide sequence of cytochrome P450 gene CYP72A61 (or its complementary chain) as primers, to a microorganism, a plant, or an animal capable of producing 22-hydroxylated oleanene triterpenes.

Alternatively, the polynucleotide may be obtained by chemical synthesis on the basis of the information of the nucleotide sequence. This method can be carried out in accordance with the descriptions in Gene, 60, 1, 115-127, 1987.

The transformant of the present invention may be prepared by introducing the gene or the recombinant vector of the present invention into an appropriate host cell.

In the present invention, a DNA encoding a polypeptide having 24-hydroxylase activity for oleanene triterpenes, or a DNA encoding a polypeptide having β-amyrin synthase activity may be introduced into a host cell. Examples of the DNA encoding a polypeptide having 24-hydroxylase activity for oleanene triterpenes include gene pESC-CYP93E1 encoding 24-hydroxylase for oleanene triterpenes (WO 2005/080572). Examples of the DNA encoding a polypeptide having β-amyrin synthase activity include β-amyrin synthase gene PSY (Accession No. AB034802, Eur. J. Biochem., 267, p. 3543-3460, 2000).

The host cell which may be used in the present invention is not limited, and any cell capable of expressing the gene of the present invention, for example, a microorganism, a yeast, a filamentous fungus, a plant cell, or an animal cell, may be used. Examples of the microorganism include gram-positive bacteria such as genus Bacillus and Streptomyces, and gram-negative bacteria such as Escherichia coli.

As the yeast, for example, strain GIL747 deficient in heme synthesis and fatty acid synthesis may be used. Strain GIL747 is a strain obtained by crossing strain GL7 [E. G. Gollub et al., J. Biol. Chem., 252, 2846-2854 (1977)] with strain W303 [Genetics 142(3):749-59]. Further, strain GIL77 deficient in lanosterol synthase ERG7 [Kushiro, T. et al., Eur. J. Biochem., 256, 238-2448 (1998)], strain INVSc1 (manufactured by Invitogen), or strain INVSc2 (manufactured by Invitogen) may be used.

As the filamentous fungus, for example, genus Aspergillus, Neurospora, Fusarium, or Trichoderma may be used. As the plant, for example, a soybean may be used. As the animal, for example, a silkworm may be used. As mammalian cells, for example, HFK293 cell, HeLa cell, COS cell, BHK cell, CHL cell, or CHO cell may be used.

The medium and culture conditions for a host cell may be appropriately selected in accordance with known methods. When a microorganism is used as the host cell, the medium to be used for culturing the obtained transformant may be either a natural medium or a synthetic medium, so long as it is a medium which contains a carbon source, a nitrogen source, inorganic salts, and the like that can be utilized by the microorganism and helps to culture the transformant efficiently.

Examples of the carbon source include potato dextrose, glucose, sucrose, soluble starch, glycerol, dextrin, molasses, and organic acids. Examples of the nitrogen source include inorganic salts (such as ammonium sulfate, ammonium carbonate, ammonium phosphate, and ammonium acetate), ammonium salts of organic acids, other nitrogen compounds, peptone, yeast extract, corn steep liquor, casein hydrolysates, and meat extract. Examples of the inorganic salts include potassium primary phosphate, potassium secondary phosphate, magnesium phosphate, magnesium sulfate, sodium chloride, ferrous sulfate, manganese sulfate, copper sulfate, and magnesium carbonate.

Transformation of a microorganism may be carried out by, for example, a protoplast method or a method using competent cells. Examples of a method for introducing a recombinant vector into a yeast as the host cell include electroporation, a spheroplast method, and a lithium acetate method. When the host cell is a silkworm, the polypeptide of the present invention can be expressed in the silkworm by, for example, a known method using a baculovirus expression system (Appl. Microbiol. Biotechol., 62, 1-20, 2003).

When a plant cell is used as the host, the transformant can be effectively prepared by, for example, Agrobacterium tumefaciens Ti plasmid or a binary vector system, Agrobacterium rhizogenes plasmid, direct gene transferring methods using polyethylene glycol, or electroporation (Methods in Molecular Biology, 267, Recombinant Gene Expression, 329-350, 2004).

The gene of the present invention may be used by inserting it into an appropriate vector. Any vector may be used in the present invention. Examples of the vector include a self-replicating vector (such as a plasmid), and a vector which is integrated into the genome of the host cell and replicated together with chromosomes when the host is transformed with the vector. An expression vector is preferable.

As the expression vector, a vector containing a promoter at the position where the polynucleotide of the present invention can be transcribed may be used. The promoter is a DNA sequence which exhibits transcription activity in a host cell, and may be appropriately selected in accordance with the type of host cell.

When the host cell is a microorganism, examples of the expression vector include pBuescript (manufactured by Stratagene), pUC (manufactured by Takara Bio Inc.), pUC118 (manufactured by Takara Bio Inc.), and pUC19 (manufactured by Takara Bio Inc.).

The promoter which may be used in the present invention is not limited, so long as it functions in a host cell such as Escherichia coli or a filamentous fungus. Examples of the promoter include a promoter derived from Escherichia coli or phages, such as trp promoter (Ptrp) or lac promoter (Plac), and a promoter derived from Aspergillus oryzae, such as Taka-amylase gene promoter or TEF1 gene promoter. Further, a promoter which is artificially designed or modified may be used.

When the host cell is a yeast, examples of the expression vector include pAUR101 (manufactured by Takara Bio Inc.), pAUR112 (manufactured by Takara Bio Inc.), pI-RED1 (manufactured by TOYOBO CO., LTD.), pYES2 (manufactured by Invitogen), pESC-URA (manufactured by Stratagene), pESC-HIS (manufactured by Stratagene), and pESC-LES (manufactured by Stratagene).

The promoter contained in the expression vector is not limited, so long as it functions in the yeast. For example, a glycolytic pathway enzyme gene promoter or a Gal promoter may be used.

The recombinant vector of the present invention may further contain a selection marker for selecting a transformant. As the selection marker, for example, a drug-resistant marker gene or an auxotrophic marker gene may be appropriately selected. When the host cell is a bacterium, for example, ampicillin resistance gene, kanamycin resistance gene, or tetracycline resistance gene may be used. When the host cell is a yeast, for example, tryptophan synthesis gene (such as TRP1), uracil synthesis gene (such as URA3), or leucine synthesis genes (such as LEU2) may be used. When the host cell is a fungus, for example, hygromycin resistance gene (Hyg), bialaphos resistance gene (Bar), or nitrate reductase gene (niaD) may be used.

The recombinant vector may be introduced by any method capable of introducing the polynucleotide into the host cell. Examples of the method include a method using calcium ions [Proc. Natl. Acad. Sci. USA, 69, 2110 (1972)], electroporation [Methods. Enzymol., 194, 182 (1990)], spheroplast method [Proc. Natl. Acad. Sci. USA, 84, 1929 (1978)], and a lithium acetate method [J. Bacteriol. 153, 163 (1983)].

The transformant of the present invention includes various embodiments, and may be prepared by, for example, transforming yeast strain GIL747 with two plasmids pESC-URA-PSY and pESC-HIS-CYP72A61. Sophoradiol may be produced using the resulting transformant. In this embodiment, P450 activity can be amplified by co-expressing soybean-derived NADPH-cytochrome P450 reductase (GmCPR) gene (Accession No. AY170374) using plasmid pESC-LEU-GmCPR.

This transformed yeast containing GmCPR was designated GIL747/pESC-URA-PSY/pESC-LEU-GmCPR/pESC-HIS-CYP72A61, and deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Jun. 30, 2009 under deposit number FERM BP-11138.

When the transformant of the present invention is used to produce soyasapogenol B, soyasapogenol B may be produced by, for example, transforming yeast strain INVSc1 with plasmids pESC-HIS-CYP72A61 and pESC-LEU-GmCPR, and adding olean-12-ene-3β,24-diol to the resulting transformant. Alternatively, soyasapogenol B may be produced by, for example, transforming yeast strain GIL747 with three plasmids pESC-URA-PSY-CYP93E1, pESC-LEU-GmCPR, and pESC-HIS-CYP72A61. That is, soyasapogenol B can be produced from β-amyrin in one step by transforming a host cell with 24-hydroxylase gene together with 22-hydroxylase gene of the present invention.

The transformed yeast was designated GIL747/pESC-URA-PSY-CYP93E1/pESC-LEU-GmCPR/pESC-HIS-CYP72A61, and deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Jun. 30, 2009 under deposit number FERM BP-11139.

Olean-12-ene-3β,24-diol may be produced by a transformant obtained by transforming yeast strain GIL77 with plasmid pESC-PSY-CYP93E1. This transformed yeast was designated GIL77/pESC-PSY-CYP93E1, and deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Feb. 6, 2004 under deposit number FERM P-19675, and was transferred to an international deposit (deposit number FERM BP-10201) on Jan. 6, 2005.

The polypeptide of the present invention may be isolated and purified from the culture broth of the transformant by known methods for the isolation and purification of enzymes.

For example, when the polypeptide of the present invention is expressed in the host cells in the soluble state, the cultivated cells are harvested by centrifugation, suspended in a buffer, disrupted with a homogenizer, sonicator, French press or the like, and centrifuged to obtain a cell-free extract. The resulting cell-free extract may be further centrifuged to obtain the supernatant, and the purified polypeptide of interest may be obtained from the supernatant by a conventional method generally-used for the isolation and purification of enzymes.

The buffer may contain an antioxidant, a stabilizer for enzymes, a polyphenol adsorbent, a ligand to metal, or the like to avoid the inactivation of the polypeptide. Further purification of the polypeptide is useful in increasing the specific activity.

Examples of the purification method include solvent extraction, salting-out using ammonium sulfate or the like, desalting, precipitation using organic solvent, anion exchange chromatography, cation exchange chromatography, hydrophobic chromatography, gel filtration, affinity chromatography, chromatofocusing, and electrophoresis such as isoelectric focusing electrophoresis. These methods may be carried out alone, or as a combination thereof.

When the polypeptide of the present invention is expressed in the host cells in the insoluble state, the cells may be disrupted in a similar fashion as described above, and centrifuged to obtain the precipitation fraction, and a conventional method may be used to collect the polypeptide of interest from the precipitation fraction. The resulting insoluble body of the polypeptide may be dissolved with a denaturing agent and, after the dilution or dialysis of the solution, the above-mentioned isolation and purification methods may be used to obtain the purified polypeptide.

When the polypeptide of the present invention is secreted to the extracellular medium, the polypeptide or a derivative thereof may be collected from the supernatant of the culture broth. The culture broth may be centrifuged in a similar fashion as described above to collect the soluble fraction, and the above-mentioned isolation and purification methods may be used to obtain the purified polypeptide from the soluble fraction.

An oleanene triterpene as a substrate and a coenzyme are added to a buffer containing the resulting polypeptide, and incubated at 15 to 40° C., preferably 20 to 37° C. As the coenzyme, NADH or NADPH may be used, and an NADPH regenerating system containing glucose 6-phosphate and glucose 6-phosphate dehydrogenase may be used together. Hydroxylation may be carried out by introducing an external gene for NADPH-P450 reductase other than the internal NADPH-P450 reductase produced by the transformant.

### EXAMPLES

The present invention now will be further illustrated by, but is by no means limited to, the following Examples.

### Example 1

### Preparation of cDNAs from Soybean Seeds

Soybean (Glycine max) seeds (Aodaizu, purchased from Toraya Sangyou Co., Ltd.) were placed on moist absorbent cotton, and germinated at 25° C. under dark conditions for 5 days. Soybean sprouts (5.3 g) were collected, frozen in liquid nitrogen, and homogenized with a mortar and pestle. The resulting homogenate was suspended in 4.5 mL of 1 mol/L Tris-HCl buffer (pH 9.0). To this suspension, 5 mL of phenol/chloroform/isoamyl alcohol (25:24:1) solution [This mixed solution was saturated by adding 1 mol/L Tris-HCl buffer (pH 9.0) thereto. Hereinafter referred to as PCI] and 0.5 mL of 10% SDS aqueous solution were added, and well mixed on ice. After 1 mL of aliquots were taken from this mixture, these aliquots were centrifuged at 15000 rpm for 5 minutes at 4° C. to collect their upper layers. After 400 μL of PCI was added to each upper layer and well mixed, the upper layers were collected under the same conditions. After these upper layers were combined, 240 μl of aliquots were taken therefrom. To each aliquot, 24 μL of 3 mol/L sodium acetate aqueous solution and 800 μL of ethanol were added, and ethanol precipitation was carried out by leaving the mixtures at −80° C. for 1 hour. These mixtures were centrifuged at 15000 rpm for 10 minutes at 4° C. The supernatants were discarded, and the pellets were dissolved and combined in 800 μL of distilled water in total. The solution was centrifuged at 15000 rpm for 10 minutes at 4° C., and the collected supernatant was divided into two tubes in a volume of 350 μL. To each tube, 350 μL of 4 mol/L lithium chloride aqueous solution was added and well mixed, and centrifuged at 15000 rpm for 10 minutes at 4° C. to discard the supernatant. To each tube, 180 μl of 70% ethanol aqueous solution was added, and centrifuged at 15000 rpm for 1 minute at 4° C. to discard the supernatant. After vacuum drying, the pellets were dissolved by adding 50 μL of distilled water to each tube to obtain total RNA (0.78 μg/μL and 1.17 μg/μL in each tube).

Reverse transcription was carried out by adding 1 μL of oligo dT primer (RACE32) to 5 μg of the resulting total RNA, using reverse transcriptase [Super Script (trademark) III, manufactured by Invitrogen], in accordance with the protocol attached thereto. The resulting product was diluted with 80 μL of distilled water to prepare a cDNA pool of soybean as a cDNA template for PCR.

RACE32:

| | |
|---|---|
| 5′-gac tcg agt cga cat cga t₁₄-3′ | (SEQ ID NO: 3) |

### Example 2

### Determination of Full Sequence of CYP72A61

Soybean CYP72A61 on the soybean EST database (http://compbio.dfci.harvard.edu/tgi/plant.html) is a partial-length TC (Tentative Consensus) sequence TC204312 lacking approximately 150 amino acid residues at the N-terminus. This sequence data is transferred to TC343128.

The partial-length TC sequence TC204312 showed 83% homology to CYP72A61 (MtCYP72A61: Accession No. DQ335793) of Medicago truncatula (Leguminosae) on the GenBank database.

Primers were designed based on the MtCYP72A61 sequence to determine the upstream sequence of TC204312. The MtCYP72A61 sequence was compared to CYP72A5 (ABCYP72A5) of Arabidopsis thaliana (Brassicaceae) with 49% homology to MtCYP72A61, to find a sequence including conserved tryptophan located at about 50 bp from the N-terminus. Because the codon for tryptophan is one, a sense primer (Mt72A61-S) was designed based on the partial MtCYP72A61 nucleotide sequence corresponding to this portion. Further, an antisense primer (GmTC204312-1st) was designed based on the sequence located at about 1100 bp upstream from the C-terminus of the TC204312 sequence. These two primers and the cDNA template prepared in Example 1 were used to amplify the desired fragment by PCR.

The resulting amplified fragment was subjected to agarose gel electrophoresis, and the agarose gel containing the desired fragment (350 to 450 bp) was excised based on a DNA molecular weight marker (Kb ladder, manufactured by Takara Bio Inc.). The desired fragment was purified from the agarose gel using MonoFas (trademark) DNA purification kit I (manufactured by GL Sciences), and eluted with 10 μL of distilled water. PCR was carried out using 1 μL of this eluate as the template together with the sense primer (Mt72A61-S) and an antisense primer (GmTC204312-2nd). After the PCR, the reaction liquid was transferred onto a Suprec 02 (trademark) filter (manufactured by Takara Bio Inc.) to remove the primers, ethanol precipitation was carried out, and the pellet was dissolved in 20 μL of distilled water. DNA Ligation Kit Ver. 2.1 (manufactured by Takara Bio Inc.) was used to ligate this fragment with pT7blue T-vector (manufactured by Novagen), and E. coli DH5α was transformed therewith and incubated on an LB (+amp, X-gal, and IPTG) plate at 37° C. for 16 hours. Grown white colonies were analyzed by colony PCR to select the desired colonies. The selected colonies were cultured in an LB (+amp) medium at 37° C. for 16 hours, and plasmids were purified from the E. coli cultures using GFX Micro Plasmid Prep kit (manufactured by GE Healthcare). These plasmids were analyzed using a capillary DNA sequencer ABI PRISM (trademark) 3100 (manufactured by Applied Biosystem) to determine a partial sequence of the upstream region at the N-terminus of TC204312.

The further upstream sequence was determined by 5′-RACE (Rapid Amplification of cDNA End). Reverse transcription was carried out using the soybean RNA prepared in Example 1 as the template, together with an antisense primer (GmTC204312-ClaI-C) designed based on the C-terminal sequence of TC204312 instead of the RACE32 primer, in accordance with the protocol attached to Super Script (trademark) III (manufactured by Invitrogen) to prepare a cDNA template for 5′-RACE. This product was used as the template together with the RACE32 primer and an antisense primer (GmTC204312-3rd) designed based on the sequence determined by the previous PCR to carry out PCR.

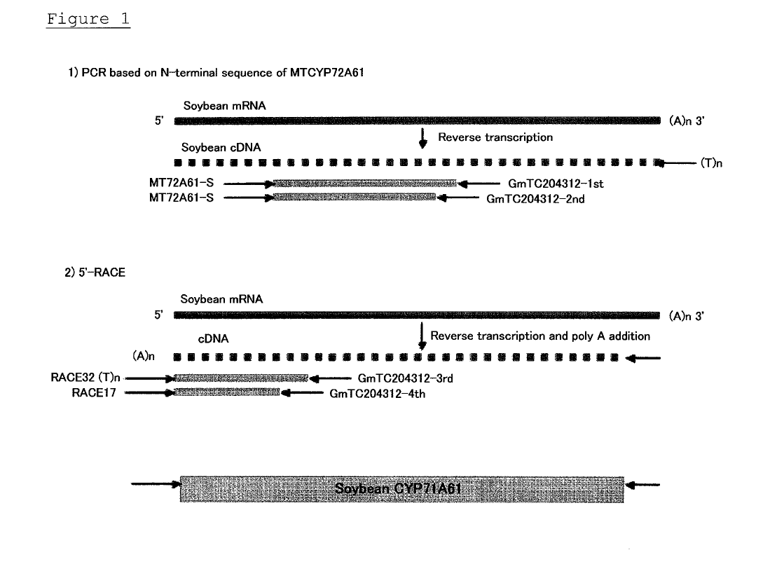

The amplified fragment was purified, and a portion of the fragment was used as the template together with a sense primer (RACE17) and an antisense primer (GmTC204312-4th) to carry out PCR. After the reaction, the reaction liquid was transferred onto a Suprec 02 (trademark) filter (manufactured by Takara Bio Inc.) to remove the primers, ethanol precipitation was carried out, and the pellet was dissolved in 10 μL of distilled water. DNA Ligation Kit Ver. 2.1 (manufactured by Takara Bio Inc.) was used to ligate this fragment with pT7blue T-vector (manufactured by Novagen), and E. coli DH5α was transformed therewith and incubated on an LB (+amp, X-gal, and IPTG) plate at 37° C. for 16 hours. Grown white colonies were analyzed by colony PCR to select the desired colonies. The selected colonies were cultured in an LB (+amp) medium at 37° C. for 16 hours, and plasmids were purified from the E. coli cultures using GFX Micro Plasmid Prep kit (manufactured by GE Healthcare). The nucleotide sequence inserted into these plasmids were analyzed using a capillary DNA sequencer ABI PRISM (trademark) 3100 (manufactured by Applied Biosystem) to determine the complete sequence of CYP72A61 (the nucleotide sequence is SEQ ID NO: 1, and the amino acid sequence is SEQ ID NO: 2.). The outline of the procedure in Example 2 is shown in FIG. 1.

Mt72A61-S:
(SEQ ID NO: 4)
5′-gtg ttg ata tgg tgg att tgg-3′
GmTC204312-1st:
(SEQ ID NO: 5)
5′-ctg tgt tta gcc cac ttg tcg cca tc-3′
GmTC204312-2nd:
(SEQ ID NO: 6)
5′-ctt gaa aag tgg act agt gtc ggg c-3′
GmTC204312-3rd:
(SEQ ID NO: 7)
5′-tcc aat caa agg gcg gta gg-3′
GmTC204312-ClaI-C:
(SEQ ID NO: 8)
5′-ggt agc tac tag agt ttg cgt aaa atg aga tga
gcc-3′
RACE17:
(SEQ ID NO: 9)
5′-gac tcg agt cga cat cg-3′
GmTC204312-4th:
(SEQ ID NO: 10)
5′-ttg aga cgc ctc tct atc ctc-3′

### Example 3

### Cloning of NADPH-Cytochrome P450 Reductase (GmCPR) Gene from Soybean

In accordance with the report of Siminszky et al. (B. Siminszky et al., Pest. Biochem. Physiol. (2003) 77, 35-43), the sequence information of soybean (Glycine max) GmCPR (Accession No. AY170374) was obtained from the GenBank database. Based on the information, an N-terminal primer (GMR-BamHI-S) including the BamHI site added to the upstream of the initiation codon and a C-terminal primer (GMR-SalI-A) including the SalI site added to the downstream of the stop codon were designed, and a fragment was amplified by PCR using the soybean cDNA template for PCR prepared in Example 1. As a result, it was confirmed by electrophoresis that the desired fragment of approximately 2 kbp was amplified.

GMR-BamHI-S:
(SEQ ID NO: 11)
5′-gtt tgt gga tcc acc atg get tcg aat tcc-3′
(The BamHI recognition site is underlined.)
GMR-SalI-A:
(SEQ ID NO: 12)
5′-taa tca gtc gac tta cca gac atc tct aag-3′
(The SalI recognition site is underlined.)

### Example 4

### Construction of Expression Plasmid pESC-HIS-GmCPR

The whole of the reaction liquid remaining after the procedure of Example 3 was applied to a Suprec 02 (trademark) filter to remove the primers from the reaction liquid, and the same volume of PCI was added to the reaction liquid and vortexed. The whole was centrifuged at 15000 rpm for 5 minutes to collect the upper layer. The amplified fragment was collected from the upper layer by ethanol precipitation, and digested with restriction enzymes BamHI and SalI, and the resulting GmCPR fragment was collected by ethanol precipitation, and inserted into the following vector pESC-HIS (manufactured by Stranagene) for expression in yeast. The vector was previously digested with restriction enzymes BamHI and SalI, collected by ethanol precipitation, end-dephosphorylated using shrimp alkaline phosphatase (manufactured by Takara Bio Inc.), and collected by ethanol precipitation. The GmCPR fragment was ligated with vector pESC-HIS using DNA Ligation Kit Ver. 2.1, and E. coli DH5α was transformed therewith and incubated on an LB (+amp) plate at 37° C. for 16 hours. Grown colonies were analyzed by colony PCR to select the desired colonies. The selected colonies were cultured in an LB (+amp) medium at 37° C. for 16 hours, and plasmids were purified from the E. coli cultures using GFX Micro Plasmid Prep kit. A portion of each plasmid solution was digested with NheI to confirm that the desired plasmid was obtained. The nucleotide sequence inserted into these plasmids were analyzed using a capillary DNA sequencer ABI PRISM (trademark) 3100 (manufactured by Applied Biosystem) to confirm that pESC-HIS-GmCPR was obtained.

### Example 5

### Construction of Expression Plasmid pESC-LEU-GmCPR

The solution of plasmid pESC-HIS-GmCPR obtained in Example 4 was digested with restriction enzymes BamHI and SalI, and the whole was applied to electrophoresis using agarose gel [Seakem (trademark) ME agarose, manufactured by Cambrex)] stained with Gel Indicator (trademark) DX (manufactured by BioDynamics Laboratory). A fragment of approximately 2 kbp corresponding to GmCPR was excised from the gel, and purified using MonoFas (trademark) DNA purification Kit I (manufactured by GL Sciences). Expression vector pESC-LEU (manufactured by Stranagene) was digested with BamHI and SalI, collected by ethanol precipitation, end-dephosphorylated using shrimp alkaline phosphatase (manufactured by Takara Bio Inc.), and collected by ethanol precipitation.

The resulting plasmid was ligated with the purified GmCPR fragment using DNA Ligation Kit Ver. 2.1 (manufactured by Takara Bio Inc.), and E. coli DH5α was transformed therewith and incubated on an LB (+amp) plate at 37° C. for 16 hours. Grown colonies were analyzed by colony PCR to select the desired colonies. The selected colonies were cultured in an LB (+amp) medium at 37° C. for 16 hours, and plasmids were purified from the E. coli cultures using GFX Micro Plasmid Prep kit (manufactured by GE Healthcare). A portion of each plasmid solution was digested with restriction enzyme ClaI. The nucleotide sequence inserted into these plasmids were analyzed using a capillary DNA sequencer ABI PRISM (trademark) 3100 (manufactured by Applied Biosystem) to confirm that pESC-LEU-GmCPR was obtained (FIG. 2).

### Example 6

### Construction of Expression Plasmid pESC-URA-PSY-CYP93E1

Expression vector pESC-URA-PSY (FIG. 2), which was prepared by inserting β-amyrin synthase gene PSY (Accession No. AB034802, Eur. J. Biochem., 267, p. 3543-3460, 2000) from pea (Pisum sativum) into the multicloning site 2 of galactose-inducible expression vector pESC-URA (manufactured by Stranagene), was introduced into E. coli DH5α, and amplified. pESC-CYP93E1 was inserted into the multicloning site 1 of the expression vector pESC-URA-PSY to obtain pESC-URA-PSY-CYP93E1. pESC-CYP93E1 was prepared in accordance with a method described in WO 2005/080572.

### Example 7

### Co-Expression of pESC-URA-PSY-CYP93E1 and pESC-HIS-GmCPR in Yeast Strain INVSc1

A case in which GmCPR obtained in Example 3 was added to a co-expression system was compared to a case in which GmCPR was not added to the co-expression system, to confirm whether or not the β-amyrin hydroxylase activity of CYP93E1 was increased. Yeast strain INVSc1 (Invitrogen) having four selective markers [uracil (URA), histidine (HIS), leucine (LEU), and tryptophan (TRP)] was used as a host.

Using Frozen-EZ Yeast Transformation II (trademark) (manufactured by ZYMO RESEARCH), strain INVSc1 was cultured on a synthetic complete agar medium without uracil and histidine containing glucose as a carbon source (SC-U-H) at 30° C. for two day, and transformed with two plasmids pESC-URA-PSY-CYP93E1 and pESC-HIS-GmCPR to obtain transformed yeast INVSc1/pESC-URA-PSY-CYP93E1/pESC-HIS-GmCPR. The SC-U medium was prepared in accordance with Methods in Yeast Genetics, A Laboratory Course manual, Cold Spring Harbar Laboratory Press, 1990.

The colony grown on the medium was inoculated into 20 mL of SC-U-H liquid medium, and cultured under shaking at 30° C. for two days. The medium was replaced with an SC-U-H-Glu liquid medium without glucose to which 20% galactose aqueous solution (final concentration: 2%) and hemin (final concentration: 13 μg/mL) were added, and cultured under shaking at 30° C. for two days. The resulting culture broth was centrifuged at 1700 rpm for 5 minutes to harvest cells. After the addition of 20% potassium hydroxide solution (containing 50% ethanol), the cell suspension was boiled for 5 minutes. Liposoluble substances were extracted using 2 mL of hexane.

The above procedure was repeated, except that a transformant was prepared using empty vector pESC-HIS instead of pESC-HIS-GmCPR, to obtain liposoluble substances.

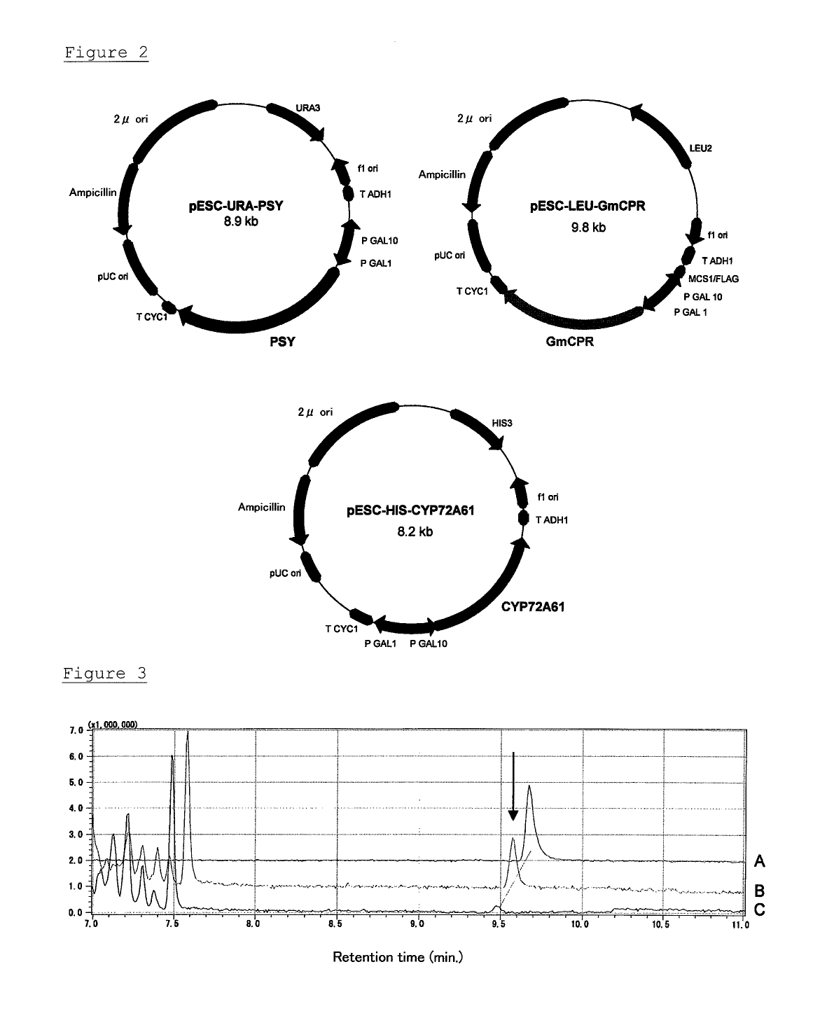

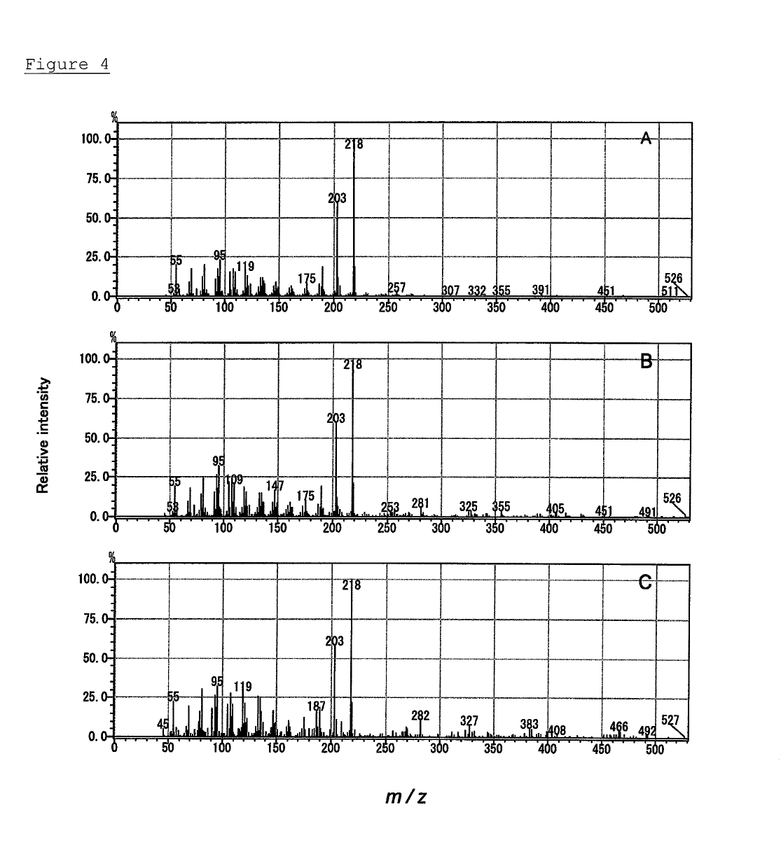

To these extracts, 100 μL of acetic anhydride and 20 μL of pyridine were added, and the mixtures were allowed to stand at room temperature for 12 hours to perform acetylation. The reaction was terminated by the addition of 100 μL of methanol and 200 μL of distilled water, and liposoluble substances were extracted twice with 2 mL of hexane. The extracted liposoluble substances were dissolved in 200 μL of acetone, and 1 μL of each acetone solution was used for GC-MS analysis. As a result, the amount of olean-12-ene-3β,24-diol produced in the transformant with GmCPR (FIG. 3-B) was apparently increased, in comparison with that in the transformant without GmCPR (FIG. 3-C), based on the total ion chromatograms. It was confirmed that the addition of GmCPR to the co-expression system increased the P450 activity (FIG. 3 and FIG. 4).

### Example 8

### Construction of Expression Plasmid pESC-HIS-CYP72A61

Based on sequence information obtained in Example 2, an N-terminal sense primer (CYP72A61-ClaI-S) including the ClaI site added to the upstream of the initiation codon and an antisense primer (CYP72A61-SacI-R) including the SacI site added to the downstream of the stop codon were designed, and a fragment was amplified by PCR using the soybean cDNA template prepared in Example 1.

CYP72A61-ClaI-S:
(SEQ ID NO: 13)
5′-gaa ttc atc gat get tat gtc tgg cac ag-3′
(The ClaI recognition site is underlined.)
CYP72A61-SacI-R:
(SEQ ID NO: 14)
5′-gaa ttc gag ctc aga gtt tgc gta aaa tg-3′
(The SacI recognition site is underlined.)

After it was confirmed by electrophoresis that the desired fragment of approximately 1600 bp was amplified, the whole of the remaining reaction liquid was applied to a Suprec 02 (trademark) filter (manufactured by Takara Bio Inc.) to remove the primers from the reaction liquid, and the same volume of PCI was added to the reaction liquid and vortexed. The whole was centrifuged at 15000 rpm for 5 minutes to collect the upper layer. The amplified fragment was collected from the upper layer by ethanol precipitation, and enzymatically digested with ClaI and SacI, and the resulting fragment was collected by ethanol precipitation to obtain a DNA insert.

Similarly, pESC-HIS was enzymatically digested with ClaI and SacI, collected by ethanol precipitation, end-dephosphorylated using shrimp alkaline phosphatase (manufactured by Takara Bio Inc.), and collected by ethanol precipitation. The resulting plasmid was ligated with the DNA insert using DNA Ligation Kit Ver. 2.1 (manufactured by Takara Bio Inc.), and E. coli DH5α was transformed therewith and incubated on an LB (+amp) plate at 37° C. for 16 hours. Grown colonies were analyzed by colony PCR to select the desired colonies. The selected colonies were cultured in an LB (+amp) medium at 37° C. for 16 hours, and plasmids were purified from the E. coli cultures using GFX Micro Plasmid Prep kit (manufactured by GE Healthcare). A portion of each plasmid solution was digested with NheI. The nucleotide sequence inserted into these plasmids were analyzed using a capillary DNA sequencer ABI PRISM (trademark) 3100 (manufactured by Applied Biosystem) to confirm that pESC-HIS-CYP72A61 was obtained (FIG. 2).

### Example 9

### Preparation of Transformant GIL747/pESC-URA-PSY/pESC-LEU-GmCPR/pESC-HIS-CYP72A61 and Identification of Extracts

A colony of strain GIL747 grown on a YPD (1% yeast extract, 2% peptone, and 2% glucose)+HET [hemin (final concentration 13 μg/mL), ergosterol (final concentration 20 μg/mL), and tween 80 (final concentration 38 mg/mL)] agar medium was inoculated into 10 mL of YPD+HET liquid medium, and cultured at 30° C. for two days under shaking (220 rpm). Frozen-EZ Yeast Transformation II (trademark) (manufactured by ZYMO RESEARCH) was used to prepare competent cells of GIL747, which were stored at −80° C. Strain GIL747 has been stored in Laboratory of Natural Products Chemistry, Graduate School of Pharmaceutical Science, The University of Tokyo, and is available to third parties.

The GIL747 competent cells were used to introduce pESC-URA-PSY into strain GIL747 in accordance with the protocol attached to Frozen-EZ Yeast Transformation II (trademark), and the cells were cultured on SC-U+HET agar medium at 30° C. for 2 days. A colony grown on this plate was inoculated into 10 mL of SC-U+HET liquid medium, and cultured at 30° C. for 2 days under shaking (220 rpm). GIL747/pESC-URA-PSY competent cells were prepared in a similar fashion, and stored at −80° C.

Further, the GIL747/pESC-URA-PSY competent cells were used to introduce pESC-LEU-GmCPR prepared in Example 5 and pESC-HIS-CYP72A61 prepared in Example 8 into GIL747/pESC-URA-PSY, and the cells were cultured on SC-U-L-H+HET agar medium at 30° C. for 3 days to prepare GIL747/pESC-URA-PSY/pESC-LEU-GmCPR/pESC-HIS-CYP72A61.

A colony grown on this agar medium was inoculated into 20 mL of SC-U-L-H+HET liquid medium, and cultured at 30° C. for 5 days under shaking (220 rpm). This liquid medium was replaced with 20 mL of SC-U-L-H+HET-Glu liquid medium, to which 2 mL of 20% galactose aqueous solution (final concentration 2%) was added, and the cells were further cultured for 24 hours under shaking. This liquid medium was replaced with 10 mL of 100 mmol/L phosphate-potassium buffer (pH 7.0), to which 100 μL of heme solution (final concentration 13 μg/mL) and 1 mL of 30% glucose aqueous solution (final concentration 3%) were added, and the cells were further cultured for 2 days under shaking. Cotton plugs were used during the sequence of cultivation so that oxygen supply did not become a rate-limiting factor. The resulting broth was centrifuged at 1700 rpm for 5 minutes to collect the cells. After the addition of 1 mL of 20% potassium hydroxide (50% ethanol) solution, the cell suspension was boiled for 5 minutes. Liposoluble substances were extracted twice with 2 mL of hexane and evaporated.

As a control, transformant GIL747/pESC-URA-PSY/pESC-LEU-GmCPR/pESC-HIS was prepared using pESC-HIS, instead of pESC-HIS-CYP72A61, to obtain liposoluble substances.

To these liposoluble substances, 50 μL of N-methyl-N-trimethylsilyl-trifluoroacetamide was added and incubated on a heat block at 80° C. for 1 hour to carry out trimethylsilylation. The resulting products were evaporated and dissolved in 200 μL of acetone, and 1 μL of aliquots were analyzed by GC-MS.

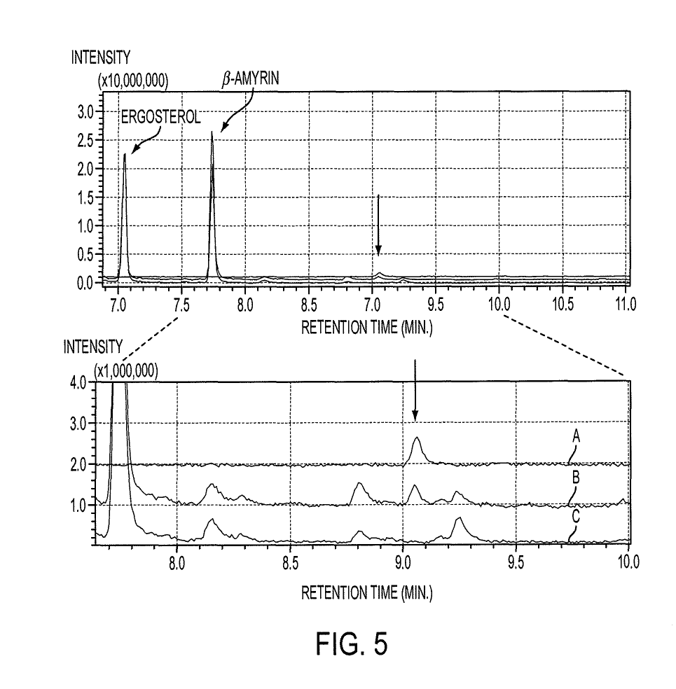

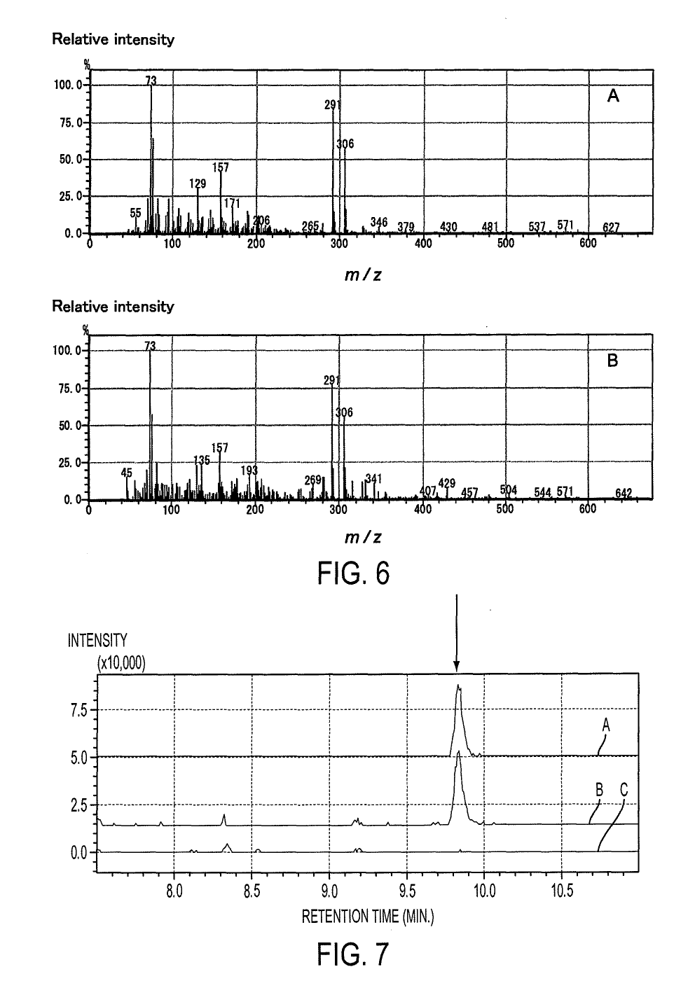

As a result, a peak which was not observed in the control (FIG. 5-C) was observed at a retention time of 9.05 minutes (FIG. 5-B) on the total ion chromatogram. As shown in FIG. 5 and FIG. 6, the retention time and the MS cleavage pattern of the peak were identical to those of the trimethylsilylation product of authentic sophoradiol (FIG. 5 and FIG. 6-A). As shown in the EI-MS cleavage patterns below, the fragment (m/z 306) is characteristic of the olean-12-ene skeleton due to retro-Diels-Alder fragmentation, and the fragment (m/z 291) was considered to be a fragment generated by the elimination of any one of the methyl groups from the fragment (m/z 306). From these results, CYP72A61 was identified as β-amyrin 22-hydroxylase.

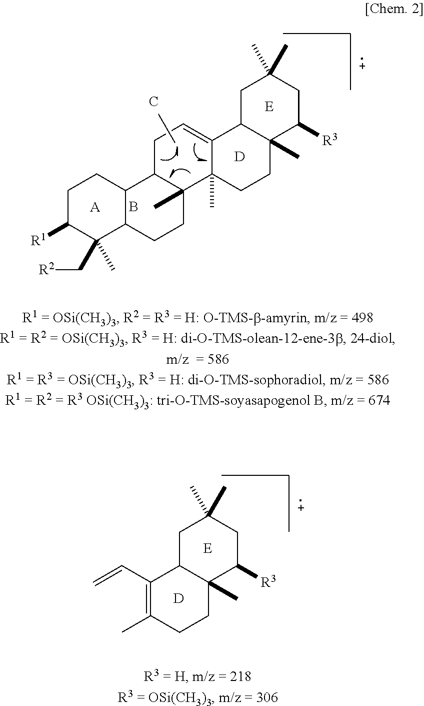

### Example 10

### Production of Substrate Olean-12-ene-3β,24-diol by 2-L Large-Scale Cultivation of GIL77/pESC-PSY-CYP93E1

Yeast strain GIL77 was transformed with pESC-PSY-CYP93E1, and cultured on SC-U+HET agar medium at 30° C. for 2 days to prepare transformed yeast GIL77/pESC-URA-PSY-CYP93E1. A colony grown on the agar medium was inoculated into SC-U+HET liquid medium, and cultured at 30° C. for 2 days. The resulting culture broth was transferred to 2 L of SCR-U+HET medium containing raffinose as a carbon source, and cultured at 30° C. for 2 days at 160 rpm. After the addition of 20% galactose aqueous solution (final concentration 2%), the cells were further cultured at 30° C. for 24 hours. The medium was replaced with 100 mmol/L phosphate-potassium buffer (pH 7.0), to which glucose (final concentration 3%) as a carbon source and hemin (final concentration 13 μg/mL) were added, and the cells were cultured at 30° C. for 24 hours. The cells were harvested by centrifugation, suspended in 200 mL of sodium hydroxide (50% methanol) solution, and heated under reflux for 1.5 hours. Liposoluble components were extracted with a mixed solvent (hexane:ethyl acetate=3:1). The resulting extract was washed with 1N hydrochloric acid, saturated sodium carbonate, and saturated sodium chloride aqueous solution. This extraction was repeated three times, and the resulting organic layers were combined, dehydrated with sodium sulfate, and evaporated. The residue was applied to flash column chromatography (Wako FC-40 silica gel), and eluted with a gradient eluent (benzene:ethyl acetate=9:1 to 4:1) to obtain 4 mg of liposoluble substance. The substance was dissolved in heavy chloroform for NMR measurement, and ¹H-NMR and ¹³C-NMR were measured. The measured values were compared with reference values [FEBS Journal, (2006) 273, 948-959] to confirm that the substance was olean-12-ene-3β,24-diol. The comparison of the chemical shift values between the measured values and the reference values [FEBS Journal, (2006) 273, 948-959] is shown in Table 1 and Table 2 (CDCl₃, ¹H: 500 MHz, ¹³C: 125 MHz).

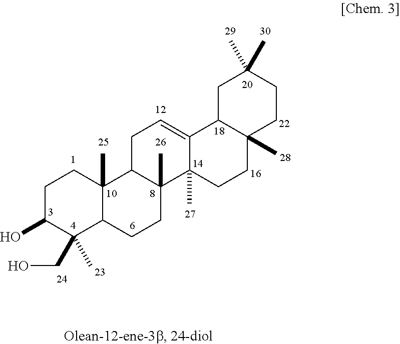

TABLE 1
¹H chemical shift values

| | Measured value | Reference value |
|---|---|---|
| Me | 0.82 | 0.82 |
| | 0.87 | 0.87 |
| | 0.87 | 0.87 |
| | 0.88 | 0.88 |
| | 0.93 | 0.93 |
| | 1.12 | 1.13 |
| | 1.25 | 1.25 |
| H-3 | 3.44 | 3.45 |
| H-12 | 5.17 | 5.17 |
| H-24 | 3.35 | 3.34 |
| | 4.20 | 4.21 |

TABLE 2
^{1 3}C chemical shift values

| Position | Measured value | Reference value |
|---|---|---|
| 1 | 38.29 | 38.30 |
| 2 | 27.64 | 27.65 |
| 3 | 80.90 | 80.91 |
| 4 | 42.75 | 42.77 |
| 5 | 55.76 | 55.77 |
| 6 | 18.42 | 18.42 |
| 7 | 32.84 | 32.84 |
| 8 | 39.75 | 39.75 |
| 9 | 47.66 | 47.67 |
| 10 | 36.62 | 36.63 |
| 11 | 23.75 | 23.75 |
| 12 | 121.52 | 121.53 |
| 13 | 145.20 | 145.20 |
| 14 | 41.65 | 41.65 |
| 15 | 26.10 | 26.10 |
| 16 | 26.86 | 26.87 |
| 17 | 32.46 | 32.46 |
| 18 | 47.18 | 47.18 |
| 19 | 46.80 | 46.81 |
| 20 | 31.08 | 31.08 |
| 21 | 34.69 | 34.69 |
| 22 | 37.08 | 37.08 |
| 23 | 22.35 | 22.34 |
| 24 | 64.50 | 64.50 |
| 25 | 16.04 | 16.03 |
| 26 | 16.68 | 16.68 |
| 27 | 25.94 | 25.94 |
| 28 | 28.38 | 28.38 |
| 29 | 23.67 | *23.67 |
| 30 | 33.33 | *33.32 |

### Example 11

### Enzyme Activity Test and Production of Soyasapogenol B Using Transformant INVSc1/pESC-LEU-GmCPR/pESC-HIS-CYP72A61

An INVSc1 colony grown on SC-L-H agar medium was inoculated into SC-L-H liquid medium, and cultured at 30° C. for 2 days under shaking (220 rpm). Frozen-EZ Yeast Transformation II (trademark) was used to prepare competent cells of INVSc1, which were stored at −80° C.

The INVSc1 competent cells were used to introduce pESC-LEU-GmCPR prepared in Example 5 and pESC-HIS-CYP72A61 prepared in Example 8 into yeast strain INVSc1 in accordance with the protocol attached to Frozen-EZ Yeast Transformation II (trademark), and the transformed cells were cultured on SC-L-H agar medium at 30° C. for 2 days to prepare transformed yeast INVSc1/pESC-LEU-GmCPR/pESC-HIS-CYP72A61.

A colony grown on the plate was inoculated into 20 mL of SC-L-H liquid medium, and cultured at 30° C. for 2 days under shaking (220 rpm). The liquid medium was replaced with 10 mL of SC-L-H-Glu liquid medium, to which 1 mL of 20% galactose (final concentration 2%) and 25 μL of 10 mmol/L olean-12-ene-3β,24-diol ethanol solution (final concentration 5 μg/mL) prepared in Example 10 were added, and the cells were further cultured at 30° C. for 4 days. The resulting culture broth was centrifuged at 1700 rpm for 5 minutes to harvest the cells. The cells were suspended in 20% potassium hydroxide (50% ethanol) solution, and boiled for 5 minutes. Extraction with 2 mL of hexane was repeated twice to obtain liposoluble components.

As a control, a transformant was prepared using empty vector pESC-HIS, instead of pESC-HIS-CYP72A61, to obtain liposoluble components.

These extracts were dissolved in acetone, and subjected to thin layer chromatography (TLC) using Silica gel 60 F₂₅₄ with concentrating zone (manufactured by MERCK). A mixture of 90 mL of benzene and 10 mL of ethyl acetate was used as a developing solvent. The developed TLC plate was irradiated with UV light to confirm the spot of ergosterol, and a portion below the spot, the portion containing high-polar compounds but not containing ergosterol, was scraped with a spatula. Liposoluble components purified on TLC were extracted with 3 mL of ethyl acetate.

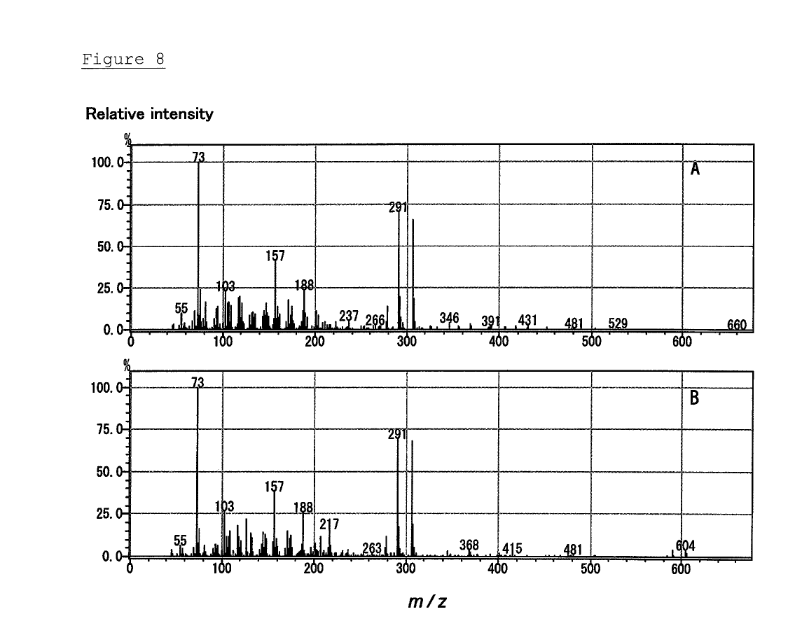

To the resulting extracts, N-methyl-N-trimethylsilyl-trifluoroacetamide was added and incubated on a heat block at 80° C. for 1 hour to carry out trimethylsilylation. The resulting products were evaporated and dissolved in 20 μL of acetone, and 1 μL of aliquots were analyzed by GC-MS. As a result, a peak (m/z 306) which was not observed in the control (FIG. 7-C) and which was characteristic of the cleavage of olean-12-ene skeleton was observed at a retention time of 9.85 minutes (FIG. 7-B). The retention time and the MS cleavage pattern of the peak were identical to those of the trimethylsilylation product of authentic soyasapogenol B (FIG. 7-A).

### Example 12

### Production of Soyasapogenol B by Co-Expression of 4 Genes of PSY, CYP93E1, GmCPR, CYP72A61

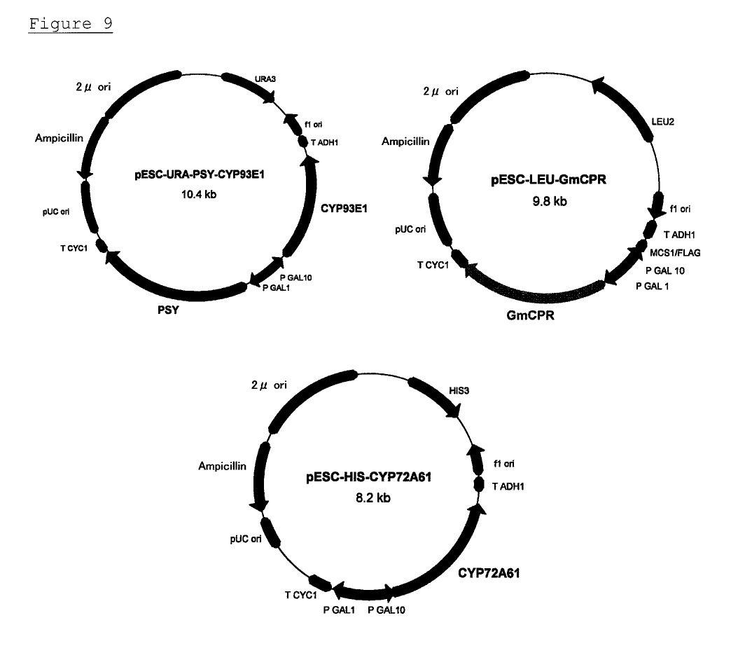

The GIL747 competent cells prepared in Example 9 were used to introduce pESC-URA-PSY-CYP93E1 and pESC-LEU-GmCPR (FIG. 9) into strain GIL747 in accordance with the protocol attached to Frozen-EZ Yeast Transformation II (trademark), and the transformed cells were cultured on SC-U-L+HET agar medium at 30° C. for 3 days. A colony grown on the plate was inoculated into 10 mL of SC-U-L+HET liquid medium, and cultured at 30° C. for 3 days under shaking (220 rpm). Competent cells of GIL747/pESC-URA-PSY-CYP93E1/pESC-LEU-GmCPR were prepared in a similar fashion, and stored at −80° C.

The resulting competent cells were transformed with pESC-HIS-CYP72A61 (FIG. 9), and cultured on SC-U-L-H+HET agar medium at 30° C. for 3 days to prepare transformed yeast GIL747/pESC-URA-PSY-CYP93E1/pESC-LEU-GmCPR/pESC-HIS-CYP72A61.

A colony grown on the agar medium was inoculated into 20 mL of SC-U-L-H+HET liquid medium, and cultured at 30° C. for 4 days under shaking (220 rpm). This medium was replaced with 20 mL of SC-U-L-H+HET-Glu liquid medium, to which 2 mL of 20% galactose aqueous solution (final concentration 2%) was added, and the cells were further cultured at 30° C. for 24 hours. This medium was replaced with 10 mL of 100 mmol/L phosphate-potassium buffer (pH 7.0), to which 1 mL of 30% glucose aqueous solution (final concentration 3%) and 100 μL of hemin (final concentration 13 μg/mL) were added, and the cells were further cultured at 30° C. for 2 days. Cotton plugs were used during the sequence of cultivation so that oxygen supply did not become a rate-limiting factor. The resulting broth was centrifuged at 1700 rpm for 5 minutes to collect the cells. After the addition of 1 mL of 20% potassium hydroxide aqueous solution (containing 50% ethanol), the cell suspension was boiled for 5 minutes. Extraction with 2 mL of hexane was repeated twice to obtain liposoluble components.

As a control, a transformant was prepared using empty vector pESC-HIS, instead of pESC-HIS-CYP72A61, to obtain liposoluble substances.

To these extracts, 50 μL of N-methyl-N-trimethylsilyl-trifluoroacetamide was added, and incubated on a heat block at 80° C. for 1 hour to carry out trimethylsilylation. The resulting products were evaporated and dissolved in 200 μL of acetone, and 1 μL of aliquots were analyzed by GC-MS.

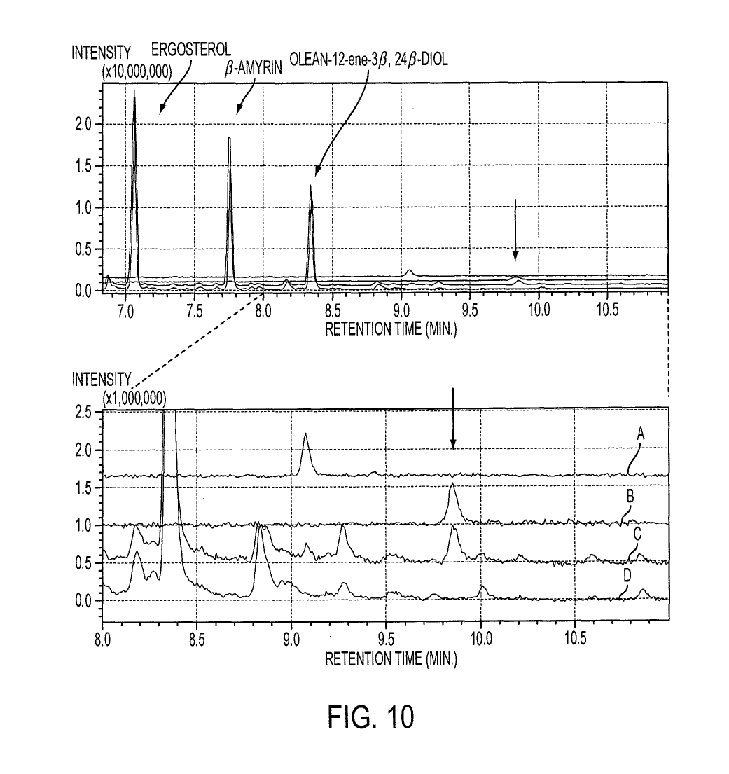

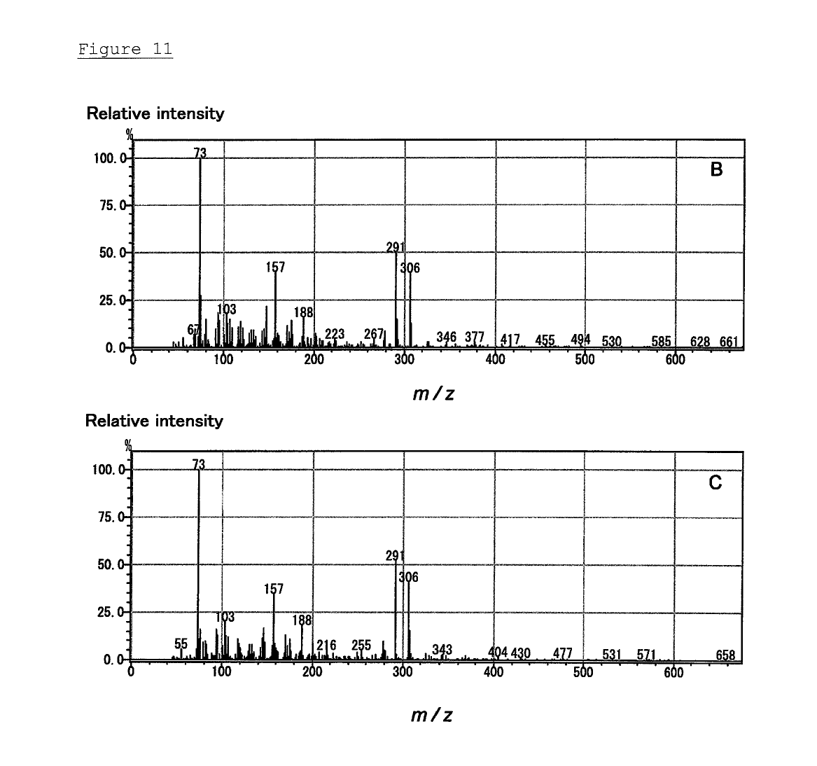

As a result, a peak which was not observed in the control (FIG. 10-D) was newly observed at a retention time of 9.85 minutes (FIG. 10-C) on total ion chromatogram. The retention time and the MS cleavage pattern of the peak were identical to those of the trimethylsilylation product of authentic soyasapogenol B (FIG. 10 and FIG. 11-B).

From these results, it was confirmed that soyasapogenol

B was produced by co-expressing 4 genes, i.e., PSY, CYP93E1, GmCPR, and CYP72A61, in a yeast expression system.

### INDUSTRIAL APPLICABILITY

According to the present invention, a hydroxylase for oleanene triterpenes at C-22 can be used by means of gene engineering, more particularly, industrial uses, for example, production of the hydroxylase using host cells transformed with a gene for the hydroxylase, or production of plant triterpenes by a microorganism. Further, triterpenes such as soyasapogenol B can be produced in large quantities by using the 22-hydroxylase gene together with a gene for 24-hydroxylase.

### Reference to Deposited Biological Material

Transformed yeast GIL747/pESC-URA-PSY/pESC-LEU-GmCPR/pESC-HIS-CYP72A61 was deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Jun. 30, 2009 under deposit number FERM BP-11138.

Transformed yeast GIL747/pESC-URA-PSY-CYP93E1/pESC-LEU-GmCPR/pESC-HIS-CYP72A61 was deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Jun. 30, 2009 under deposit number FERM BP-11139.

Transformed yeast GIL77/pESC-PSY-CYP93E1 was deposited in the International Patent Organism Depositary National Institute of Advanced Industrial Science and Technology (Address: AIST Tsukuba Central 6, 1-1, Higashi 1-chome Tukuba-shi, Ibaraki-ken 305-8566 Japan) on Feb. 6, 2004 under deposit number FERM P-19675, and was transferred to an international deposit (deposit number FERM BP-10201) on Jan. 6, 2005.

### Free Text in Sequence Listing

Features of “Artificial Sequence” are described in the numeric identifier <223> in the Sequence Listing.

Each nucleotide sequence of SEQ ID NOS: 3 to 14 is a primer sequence.

1. A polynucleotide encoding a polypeptide having 22-hydroxylase activity for an oleanene triterpene.

2. The polynucleotide according to claim 1, which is a DNA selected from the group consisting of:
(a) a DNA encoding a polypeptide comprising the amino acid sequence of SEQ ID NO: 2;
(b) a DNA comprising the coding region in the nucleotide sequence of SEQ ID NO: 1;
(c) a DNA encoding a polypeptide comprising an amino acid sequence with at least 80% identity to SEQ ID NO: 2, wherein the polypeptide has 22-hydroxylase activity for an oleanene triterpene;
(d) a DNA comprising a nucleotide sequence with at least 80% identity to SEQ ID NO: 1, wherein a polypeptide encoded by the nucleotide sequence has 22-hydroxylase activity for an oleanene triterpene; and
(e) a DNA which hybridizes under stringent conditions to a DNA consisting of the nucleotide sequence of SEQ ID NO: 1, and encodes a polypeptide having 22-hydroxylase activity for an oleanene triterpene.

3. A recombinant vector comprising the polynucleotide according to claim 1.

4. A transformant transformed with the recombinant vector according to claim 3.

5. The transformant according to claim 4, which is a yeast.

6. A polypeptide selected from the group consisting of:
(a) a polypeptide comprising the amino acid sequence of SEQ ID NO: 2;
(b) a polypeptide comprising the amino acid sequence encoded by the nucleotide sequence of SEQ ID NO: 1;
(c) a polypeptide comprising an amino acid sequence with at least 80% identity to SEQ ID NO: 2, wherein the polypeptide has 22-hydroxylase activity for an oleanene triterpene;
(d) a polypeptide comprising an amino acid sequence encoded by a nucleotide sequence with at least 80% identity to SEQ ID NO: 1, wherein the polypeptide has 22-hydroxylase activity for an oleanene triterpene; and
(e) a polypeptide encoded by a DNA which hybridizes under stringent conditions to a DNA consisting of the nucleotide sequence of SEQ ID NO: 1, wherein the polypeptide has 22-hydroxylase activity for an oleanene triterpene.

7. A process of manufacturing sophoradiol, comprising the steps of:
preparing a first recombinant vector comprising the polynucleotide according to claim 1, and a second recombinant vector comprising a DNA encoding a polypeptide having β-amyrin synthase activity,
co-expressing the two recombinant vectors in a same host cell,
cultivating the expressed host cell, and
purifying sophoradiol from the host cell or a culture supernatant.

8. The method according to claim 7, wherein a third recombinant vector comprising a DNA encoding a polypeptide having cytochrome P450 reductase activity is further expressed in the same host cell.

9. The method according to claim 7, wherein the host cell is a yeast.

10. A process of manufacturing soyasapogenol B, comprising the steps of:
preparing a first recombinant vector comprising the polynucleotide according to claim 1, a second recombinant vector comprising a DNA encoding a polypeptide having β-amyrin synthase activity, and a third recombinant vector comprising a DNA encoding a polypeptide having 24-hydroxylase activity for an oleanene triterpene, co-expressing the three recombinant vectors in a same host cell,
cultivating the expressed host cell, and
purifying soyasapogenol B from the host cell or a culture supernatant.

11. The method according to claim 10, wherein a fourth recombinant vector comprising a DNA encoding a polypeptide having cytochrome P450 reductase activity is further expressed in the same host cell.

12. The method according to claim 10, wherein the host cell is a yeast.


SEQUENCE LISTING (14 sequences)

SEQ ID NO: 1 - DNA, 1602 bp, Glycine max
  CDS (1)..(1602)
atgcttatgtctggcacagaacaggtgaccatgggaggattaggactcaatttgaccccaattactacatttgctatcattactgtgattgctacggtgctaatatggtggttttggaatgcactcaactgggtgtggctgagacccaagaggatagagaggcgtctcaaggagcagggtatccaaggaaattcctaccgccctttgattggagacatcagagatatggttaagatgattaaggaagccaaatccaaacctatggatcctcactctaatgacattgcgccccgtgtgttgccttacgttgttcacaccatcgctaaatacggtaagagttcatttatgtggcttggtccaacaccaagggtattcatcttggatcctgacaaattcaaagaaatggctactaaggtctatgattttcaaaagcccgacactagtccacttttcaagcttctagcatcagggtttgcaaattatgatggcgacaagtgggctaaacacagaaagatcgtgagtccagcattcaatgtagagaaaatgaagctcttggtaccgatattttgccagagttgcgatgatttgatcagcaaatgggagagtttgttatcttcgtctaatgggtcttgtgagttagatgtgtggccttttgtccaaaacgtgtcaagcgacgttcttgctcgtgcaggctttggaagtagctatcaagaagggaaaaaaatattcgaacttcaaagggaaatgattcagcttacaatgacgctctttaagtttgctttcattccaggttacaggtttctgccaacgcataccaacaggaggatgaaagcaattgacaaagaaatacgagaatcacttatggtaatcatcaaccgaagattaaaagcaatcaaagcaggggagcctactaacaatgacttgttaggcatactcttggaatcaaattacaaggaatctgaaaaaagtagtggtggaggaatgagtttaagggaagtagtggaagaagtgaagctattttacttggcagggcaggaagcaaatgcagaattgctggtctggacattgttgttattaagcaggcatccagattggcaagaaaaggctagggaggaggttttccaagtgttcgggaatgaaaagccagattatgaaaggattggtcaactgaaaattgtgtcgatgattctacaggagagtctcagattatacccaccagtggttatgttcgctcggtatcttcgtaaagatacaaaacttggagagcttacaattcccgcaggagtggagcttgttgtacctgtgtcaatgctacaccaggataaggagttttggggagatgatgccggggaattcaacccagaaagattctcggaaggagtatcaaaggcaacaaaaggcaagctttcctacttgccatttggatggggtcctcgactctgcataggacaaaactttggcttgttagaagcaaaagtagctgtgtcaatgatcctgcaacgtttctccctccatttttccccctcctatgctcatgccccctcctttattatcactcttcagcctgagcgtggggctcatctcattttacgcaaactctag

SEQ ID NO: 2 - protein, 533 aa, Glycine max
MLMSGTEQVTMGGLGLNLTPITTFAIITVIATVLIWWFWNALNWVWLRPKRIERRLKEQGIQGNSYRPLIGDIRDMVKMIKEAKSKPMDPHSNDIAPRVLPYVVHTIAKYGKSSFMWLGPTPRVFILDPDKFKEMATKVYDFQKPDTSPLFKLLASGFANYDGDKWAKHRKIVSPAFNVEKMKLLVPIFCQSCDDLISKWESLLSSSNGSCELDVWPFVQNVSSDVLARAGFGSSYQEGKKIFELQREMIQLTMTLFKFAFIPGYRFLPTHTNRRMKAIDKEIRESLMVIINRRLKAIKAGEPTNNDLLGILLESNYKESEKSSGGGMSLREVVEEVKLFYLAGQEANAELLVWTLLLLSRHPDWQEKAREEVFQVFGNEKPDYERIGQLKIVSMILQESLRLYPPVVMFARYLRKDTKLGELTIPAGVELVVPVSMLHQDKEFWGDDAGEFNPERFSEGVSKATKGKLSYLPFGWGPRLCIGQNFGLLEAKVAVSMILQRFSLHFSPSYAHAPSFIITLQPERGAHLILRKL

SEQ ID NO: 3 - DNA, 32 bp, Artificial Sequence
  Description of Artificial Sequence primer sequence
gactcgagtcgacatcgatttttttttttttt

SEQ ID NO: 4 - DNA, 21 bp, Artificial Sequence
  Description of Artificial Sequence primer sequence
gtgttgatatggtggatttgg

SEQ ID NO: 5 - DNA, 26 bp, Artificial Sequence
  Description of Artificial Sequence primer sequence
ctgtgtttagcccacttgtcgccatc

SEQ ID NO: 6 - DNA, 25 bp, Artificial Sequence
  Description of Artificial Sequence primer sequence
cttgaaaagtggactagtgtcgggc

SEQ ID NO: 7 - DNA, 20 bp, Artificial Sequence
  Description of Artificial Sequence primer sequence
tccaatcaaagggcggtagg

SEQ ID NO: 8 - DNA, 36 bp, Artificial Sequence
  Description of Artificial Sequence primer sequence
ggtagctactagagtttgcgtaaaatgagatgagcc

SEQ ID NO: 9 - DNA, 17 bp, Artificial Sequence
  Description of Artificial Sequence primer sequence
gactcgagtcgacatcg

SEQ ID NO: 10 - DNA, 21 bp, Artificial Sequence
  Description of Artificial Sequence primer sequence
ttgagacgcctctctatcctc

SEQ ID NO: 11 - DNA, 30 bp, Artificial Sequence
  Description of Artificial Sequence primer sequence
gtttgtggatccaccatggcttcgaattcc

SEQ ID NO: 12 - DNA, 30 bp, Artificial Sequence
  Description of Artificial Sequence primer sequence
taatcagtcgacttaccagacatctctaag

SEQ ID NO: 13 - DNA, 29 bp, Artificial Sequence
  Description of Artificial Sequence primer sequence
gaattcatcgatgcttatgtctggcacag

SEQ ID NO: 14 - DNA, 29 bp, Artificial Sequence
  Description of Artificial Sequence primer sequence
gaattcgagctcagagtttgcgtaaaatg